(19) United States
(12) Patent Application Publication
(10) Pub. No.: US 2009/0036507 A1
(43) Pub. Date: Feb. 5, 2009

(54) BIS (CYANOPHENYL) METHYL-TRIAZOLE FOR USE IN PREVENTION OF BREAST CANCER

(76) Inventor: Rajeshwar Rao Tekmal, Helotes, TX (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/246,857

(22) Filed: Oct. 7, 2008

Related U.S. Application Data
(63) Continuation of application No. 10/497,407, filed on Jan. 4, 2005, now abandoned.
(60) Provisional application No. 60/339,574, filed on Dec. 11, 2001.

(51) Int. Cl. A61K 31/4196; C07D 249/08

(52) U.S. Cl. 514/383; 548/267.4

(57) ABSTRACT

The present invention relates to the use of a lower dosage of bis(cyanophenyl)methyl-triazole of the formula (I) or a pharmaceutically acceptable salts thereof, for the preparation of a pharmaceutical composition for the prevention of conditions responsive to aromatase inhibition and to inhibition of estrogen biosynthesis, especially breast cancer, in mammals.

The present invention relates to the use of bis(cyanophenyl)methyl-triazole of the formula (I):

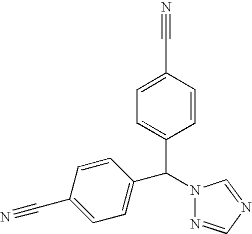

(hereinafter: “COMPOUND I”) or a pharmaceutically acceptable salts thereof, for the preparation of a pharmaceutical composition for the prevention of conditions responsive to aromatase inhibition and to inhibition of estrogen biosynthesis in mammals.

COMPOUND I is also known as Letrozole [International Nonproprietary Name].

COMPOUND I is, and has been specifically described in the European patent No. 0 236 940 published on Sep. 16, 1987, as well as in U.S. Pat. No. 4,978,672 published on Dec. 18, 1990, and Japanese Patent No. 2018112 all in the name of the applicant

COMPOUND I was originally identified as an inhibitor of aromatase.

Aromatase is the enzyme complex responsible for the final step in estrogen synthesis, viz the conversion of the androgens androstenedione and testosterone to the estrogens estrone (E₁) and estradiol (E₂). There are substantial data showing that estrogen promotes breast cancer. Increasingly, the female breast has Itself been recognized as another important site of estrogen production. Stromal cells in breast adipose tissue produce estrogen that is biologically active in both a paracrine and an autocrine manner. This is probably responsible for the observation that estrogen concentrations in the healthy breasts of postmenopausal women are unexpectedly higher (four- to six-fold) than in serum and similar to those in premenopausal women. In addition up to 70% of breast cancer cells have been shown to synthesize estrogen as a result of intracellular aromatase expression. This explains why aromatase expression and activity are higher in breast tumors than in peritumoral fat and in tumor-bearing quadrants of the breast compared with those without tumors. There is increasing evidence that this local estrogen production may play a major role in tumor proliferation. Inhibiting estrogen at the source of its synthesis is therefore a logical target of breast cancer treatment.

COMPOUND I is already known as an inhibitor of aromatase and useful for the treatment of breast cancer.

It has now surprisingly been found that COMPOUND I possesses therapeutic properties, which render it particularly useful for the prevention of conditions responsive to aromatase inhibition and to inhibition of estrogen biosynthesis in mammals. This compound shows an unexpected high potency to eliminate estrogen-mediated initiation of breast cancer. Particularly unexpected is that at lower dosage this compound still shows high potency to eliminate estrogen-mediated initiation of breast cancer without affecting normal physiologic functions. Indeed, it is particularly surprising that the use of a dosage of COMPOUND I that has no significant effect on the circulating level of estradiol and or circulating level of follicle stimulating hormone in the mammal, shows high potency to eliminate estrogen-mediated initiation of breast cancer.

The present invention thus concerns the use of COMPOUND I for the preparation of a pharmaceutical composition for the prevention of conditions responsive to aromatase inhibition and to inhibition of estrogen biosynthesis in mammals.

The present invention more particularly concerns the use of COMPOUND I for the preparation of a pharmaceutical composition for inhibiting the development of breast cancer in woman, especially in a post-menopausal woman or a woman predisposed to breast cancer.

The present invention more particularly concerns the use of COMPOUND I for the preparation of a pharmaceutical composition for inhibiting the development of breast cancer in woman, especially in a post-menopausal woman or a woman predisposed to breast cancer, characterized in that the dosage or daily dosage of COMPOUND I has no significant effect on the circulating level of estradiol and/or circulating level of follicle stimulating hormone in the mammal.

The present invention more particularly concerns the use of COMPOUND I for the preparation of a pharmaceutical composition in a unit dosage form containing 0.1 to 0.3 mg, preferably 0.125 to 0.25 mg of COMPOUND I.

The present invention more particularly concerns the use of COMPOUND I for the preparation of a pharmaceutical composition in a unit dosage form containing an amount of COMPOUND I which has no significant effect on the circulating level of estradiol and or circulating level of follicle stimulating hormone in the mammal.

The present invention in a second preferred embodiment concerns the use of COMPOUND I for the preparation of a pharmaceutical composition in a unit dosage form containing 0.001 to 0.11 mg, preferably 0.001 to 0.099 mg or 0.001 to 0.09 mg, most preferably 0.002 to 0.02 mg of COMPOUND I.

In still another embodiment, the instant invention provides a method for preventing conditions responsive to aromatase inhibition and to inhibition of estrogen biosynthesis in mammals comprising administering to a mammal in need of such treatment a therapeutically effective amount of COMPOUND I, or a pharmaceutically acceptable salts thereof.

In still another embodiment, the instant invention provides a method for preventing conditions responsive to aromatase inhibition and to inhibition of estrogen biosynthesis in mammals comprising administering to a mammal in need of such treatment a therapeutically effective amount of COMPOUND I, or a pharmaceutically acceptable salts thereof, wherein the therapeutically effective amount of COMPOUND I has no significant effect on the circulating level of estradiol and or circulating level of follicle stimulating hormone in the mammal.

Preferably the mammal is a human, especially a post-menopausal woman or a woman predisposed to breast cancer.

Preferably, this method is used with a daily dosage of COMPOUND I, 8.3 to 25 fold lower than the daily dosage necessary for the curative treatment of conditions responsive to aromatase inhibition and to inhibition of estrogen biosynthesis in mammals. Good results have been obtained with a 10 to 20 fold lower dosage and the best results have been obtained with 10 to 16.7 fold lower dosage than the daily dosage necessary for the curative treatment of such conditions.

Preferably, this method is used for inhibiting the development of breast cancer.

In a preferred embodiment, the daily effective amount of COMPOUND I for inhibiting the development of breast cancer In a woman, is 0.1 to 0.3 mg, preferably 0.125 mg to 0.25 mg most preferably 0.15 mg to 0.25 mg.

In a second preferred embodiment, the daily effective amount of COMPOUND I for inhibiting the development of breast cancer in a woman, is 0.001 to 0.11 mg, preferably 0.001 to 0.099 mg or 0.001 to 0.09 mg, most preferably 0.002 to 0.02 mg.

In a most preferred embodiment, the daily effective amount of COMPOUND I for inhibiting the development of breast cancer in a woman, has no significant effect on the circulating level of estradiol and or circulating level of follicle stimulating hormone in the treated woman.

Preferably, the COMPOUND I is administered to the human subject over at least a three week time period on only about 14.2% to about 42.8% of the days in the time period.

Preferably, the COMPOUND I is administered to the human subject 1 or 2 or 3 times a week on alternate days for a period of three weeks or longer.

Preferably, the weekly dosage regimen is carried out for a period of 3, 4, 5, 6, 7, or 8 weeks. Each treatment period may be followed by a period of one to three weeks, e.g. two weeks, wherein the agent is not administered. This cycle is repeated for from 1 to several cycles, for example, from 3 or 4 to 8 or more cycles. Preferably, the weekly dosage regimen is carried out in order that there is no significant effect on the circulating level of estradiol and or circulating level of follicle stimulating hormone in the patient, during the treatment period.

In an alternative method of treatment, the COMPOUND I is administered to the human subject 7 to 4 times a week or about 100% to about 50% of the days in the time period, on alternate days, i.e daily or once each two days, for a period of from one to three weeks, e.g. two weeks, followed by a period of one to three weeks, e.g. two weeks, wherein the agent is not administered. This cycle is repeated for from 1 to several cycles, for example, from 3 or 4 to 8 or more cycles. Preferably, in the method of treatment, dosage regimen is carried out in order that there is no significant effect on the circulating level of estradiol and or circulating level of follicle stimulating hormone in the patient, during the treatment period.

A preferred example is an administration of 0.01 to 0.04 mg preferably 0.02 mg once each two days. A second preferred example is an administration of 0.001 to 0.004 mg preferably 0.002 mg daily.

In a third aspect of the invention, there is provided an article of manufacture comprising packaging material, and COMPOUND I, or a pharmaceutically-acceptable salt thereof, contained within said packaging material, wherein said packaging material comprises label directions which indicate that said COMPOUND I, or said pharmaceutically-acceptable salt of COMPOUND I, is to be administered to a woman especially a post-menopausal woman, in an amount from about 0.1 mg to about 0.3 mg preferably from about 0.125 mg to about 0.25 mg following a specific dosage regimen to inhibit the development of breast cancer. In a second preferred embodiment, the effective amount of COMPOUND I for inhibiting the development of breast cancer in a woman, is 0.001 to 0.11 mg, preferably 0.001 to 0.099 mg or 0.001 to 0.09 mg, most preferably 0.002 to 0.02 mg.

In a further embodiment, the instant invention provides a pharmaceutical composition containing an amount of COMPOUND I, 8.3 to 25 fold lower than the daily dosage necessary for the preventive treatment of conditions responsive to aromatase inhibition and to inhibition of estrogen biosynthesis in mammals. The best results have been observed with a 10 to 20 fold preferably 10 to 16.7 fold lower daily dosage.

In another aspect of the invention, there is provided an article of manufacture comprising packaging material, and COMPOUND I, or a pharmaceutically-acceptable salt thereof, contained within said packaging material, wherein said packaging material comprises label directions which indicate that said COMPOUND I, or said pharmaceutically-acceptable salt of COMPOUND I, is to be administered to a woman especially a post-menopausal woman, in an amount which has no significant effect on the circulating level of estradiol and or circulating level of follicle stimulating hormone in the mammal, following a specific dosage regimen to inhibit the development of breast cancer.

In a preferred embodiment, the instant invention provides a pharmaceutical composition in a unit dosage form containing 0.1 to 0.3 mg, preferably 0.125 to 0.25 mg of COMPOUND I, for the preventive treatment of conditions responsive to aromatase inhibition and to inhibition of estrogen biosynthesis in woman.

In a most preferred embodiment, the instant invention provides a pharmaceutical composition containing 0.15 mg to 0.25 mg of COMPOUND I, for the preventive treatment of conditions responsive to aromatase inhibition and to inhibition of estrogen biosynthesis in humans.

In a second preferred embodiment, the instant invention provides a pharmaceutical composition containing 0.001 to 0.11 mg, preferably 0.001 to 0.099 mg or 0.001 to 0.09 mg, most preferably 0.002 to 0.02 mg of COMPOUND I, for the preventive treatment of conditions responsive to aromatase inhibition and to inhibition of estrogen biosynthesis in humans.

Preferably, a pharmaceutical composition containing 0.125 to 0.3 mg or 0.002 to 0.02 of COMPOUND I, for the preventive treatment of breast cancer in woman.

In a most preferred embodiment, the instant invention provides a pharmaceutical composition in a unit dosage form containing an amount of COMPOUND I which has no significant effect on the circulating level of estradiol and or circulating level of follicle stimulating hormone in the mammal, for the preventive treatment of breast cancer.

In previous studies it has been shown that by using 5 μg COMPOUND I/mouse/daily, a dose which was based on previous animal studies we could completely abrogate the aromatase-induced preneoplastic/neoplastic changes. However, the chosen dose was not only very effective in abrogating aromatase-induced premalignant changes, but also affected some normal endocrinological functions in these animals. Current findings indicate using low dose(s) of COMPOUND I it is possible to eliminate estrogen-mediated initiation of breast cancer in transgenic mice without affecting normal physiologic functions. Our data clearly demonstrates that COMPOUND I, even when used in lower doses as described herein is very effective in abrogating aromatase-induced breast hyperplasia. Our data shows that we could completely abrogate aromatase-induced preneoplastic changes by using a low dose of COMPOUND I (0.25, 0.5 μg or 1 μg per day per animal preferably 0.25 and 0.5 μg) with no effect on histo-morphological features of the uterus and ovary.

These unexpected good results have been obtained by studying effects of COMPOUND I on assays described herein;

In particular the resulting data show that we could completely abrogate aromatase-induced preneoplastic changes by using a low dose of COMPOUND I (0.25 or 0.5 μg per day per animal). These doses had no effect on uterus and ovary. No significance difference between control (12.5 pg/ml) and the individual groups treated with low doses of COMPOUND I (0.25 or 0.5 μg per day per animal) in circulating estrogen levels (respectively 12.1 and 12.5 pg/ml). No significance difference between control and the animals treated with 0.5 μg of COMPOUND I in circulating FSH levels (200 ng/ml). We have also noticed no significant change in the levels of PCNA and cyclin D1, also good markers of estrogen-mediated proliferation with low doses of COMPOUND I (0.25 or 0.5 μg per day per animal). Which is consistent with the observation that low doses of COMPOUND I (0.25 or 0.5 μg per day per animal) had no effect on histo-morphological features of the uterus and ovary. The expression of the uterine marker lactoferrin showed no difference between the control and the 0.25- and 0.5 μg-treated animals.

These studies suggest that using very low concentrations of an aromatase inhibitor it is possible to eliminate estrogen mediated initiation of breast cancer in transgenic mice without affecting normal uterine and ovarian function.

Estrogen and other hormone levels in the blood are normally determined using ELISA (Enzyme Linked ImmunoSorbent Assay) or RIA (Radio Immuno Assay) that are available from various providers and well known in the art. More sensitive and more sophisticated and therefore not routinely used are HPLC (High performance Liquid Chromatography) or mass spectroscopy.

Thus, the COMPOUND I is shown to be very highly suitable for the prevention of conditions responsive to aromatase inhibition and to inhibition of estrogen biosynthesis in mammals especially with a daily dosage 8.3 to 25 fold lower than the daily dosage necessary for the curative treatment of conditions responsive to aromatase inhibition and to inhibition of estrogen biosynthesis in mammals. The best results have been observed with a 10 to 20 fold preferably 10 to 16.7 fold lower daily dosage.

Thus, the COMPOUND I is shown to be very highly suitable for the prevention of conditions responsive to aromatase inhibition in mammals especially with a dosage or daily dosage that has no significant effect on the circulating level of estradiol and or circulating level of follicle stimulating hormone in the mammal.

The term “conditions responsive to aromatase inhibition and to inhibition of estrogen biosynthesis” as used herein includes, but is not limited to, breast cancer or breast adenocarcinoma.

The term “no significant effect on the circulating level of estradiol and or circulating level of follicle stimulating hormone in the mammal” as used herein means that there is no significant difference in the estradiol and or follicle-stimulating hormone circulating level between the patient not treated with COMPOUND I and the treated patient.

The term “no significant effect” as used herein means that the estradiol and or follicle stimulating hormone circulating level does not change more than 15%, preferably 10%, most preferably 5%, with respect to the control level before treatment.

Breast adenocarcinoma or cancer is a major medical problem in women, particularly above the age of thirty-five. Currently, it is estimated that women have a one in nine chance of developing this disease in the course of their lifetime. Breast carcinoma is a major cause of mortality in women, as well as a cause of disability, psychological trauma, and economic loss. A large number of women contracting this disease eventually die from its effects either directly or indirectly from complications.

The term “curative” as used herein means efficacy in treating persistent conditions responsive to aromatase inhibition and to inhibition of estrogen biosynthesis in mammals.

In the present description, the term “prevention” includes treatment of patients at risk of contracting the disease or suspected to have contracted the disease in order to avoid the onset or recurrence of conditions responsive to aromatase inhibition and to inhibition of estrogen biosynthesis (i.e. inhibiting the development of breast cancer).

The term “inhibiting the development of breast cancer” as used herein is intended to primarily refer to a situation in which de novo transformation of normal breast cells to cancerous or malignant cells is inhibited. However, there may be situations in which women have clinically non-detectable cancerous cells in their breasts, and the inhibition of development of such, as yet, clinically insignificant cancers also forms part of the invention. Not included within the scope of this Invention, is the therapy of existing, clinically-detectable breast cancer.

The term “woman predisposed to breast cancer” relates to woman responding to some specific criteria well known by the physican such as the age (at this time, age is a major identifiable risk factor. More than 80% of breast cancer cases occur in women over 50), ethnicity, genetic factors and family history (an estimated 10% of all women with breast cancer have a strong family history of the disease, which often appears in young women under the age of 50—Inherited mutations in genes known as BRCA1 or BRCA2 are now believed to be responsible for 30% to 50% of breast cancers), over-exposure to estrogen (because growth of breast tissue is highly sensitive to estrogens, the more a women is exposed to estrogen over her lifetime, the higher the risk for breast cancer), physical characteristics, certain breast abnormalities.

To demonstrate that COMPOUND I is particularly suitable for the prevention of conditions responsive to aromatase inhibition and to inhibition of estrogen biosynthesis, with good therapeutic margin and other advantages, clinical trials can be carried out in a manner known to the skilled person.

The dosage of COMPOUND I currently employed for the curative treatment of conditions responsive to aromatase inhibition and to inhibition of estrogen biosynthesis in woman is a daily oral dosage of 2.5 mg.

The precise dosage of COMPOUND I to be employed for the prevention of conditions responsive to aromatase inhibition and to inhibition of estrogen biosynthesis, depends upon several factors including the host, the nature and the severity of the condition being treated, the mode of administration. However, satisfactory prevention may be achieved when the COMPOUND I is administered (check preferred administration) parenterally, e.g., intraperitoneally, intravenously, intramuscularly, subcutaneously, intratumorally, or rectally, or enterally, e.g., orally, at a daily dosage of 0.1 to 0.3 mg, preferably 0.125 to 0.25 mg in humans. Most preferably in a dosage or daily dosage that has no significant effect on the circulating level of estradiol and or circulating level of follicle stimulating hormone in the mammal.

Most preferably, the COMPOUND I is administered orally, by dosage forms such as microemulsions, soft gels or solid dispersions in dosages up to 0.1 to 0.25 mg/day, administered up to three times a day. Most preferably in a dosage or daily dosage that has no significant effect on the circulating level of estradiol and or circulating level of follicle stimulating hormone in the mammal.

The upper limit of dosage is that imposed by side effects and can be determined by trial for the host being treated.

A preventive treatment is often a long period treatment and the treatment regimen should therefore be adapted to reduce risks of accumulation of COMPOUND I in the body and the subsequent elevation of liver enzymes which might occur few weeks after the initiation of the treatment. An adapted treatment regimen should also avoid the development of resistance against the pharmacological action of COMPOUND I, or the lowering of its pharmacological efficacy in the host being treated.

In a further aspect, to reduce the above mentioned risks, the present invention relates to a treatment regimen whereby over at least a three week period, the COMPOUND I is administered on only about 14.2% to about 42.8% of the days.

Alternatively, the COMPOUND I is administered to the human subject 7 to 4 times a week or about 100% to about 50% of the days in the time period, on alternate days, i.e. daily or once each two days, of from one to three weeks, e.g. two weeks, followed by a period of one to three weeks, e.g. two weeks, wherein the agent is not administered. This cycle is repeated for from 1 to several cycles, for example, from 3 or 4 to 8 or more cycles.

In the described treatment regimens or articles of manufacture the effective amount of bis(cyanophenyl)methyl-triazole can be in the form of a pharmaceutical composition as described herein.

COMPOUND I is administered in a unit dosage form containing 0.1 to 0.3 mg, preferably 0.125 to 0.25 mg, most preferably 0.15 to 0.25 mg. Most preferably in a unit dosage form that has no significant effect on the circulating level of estradiol and or circulating level of follicle stimulating hormone in the mammal.

As used herein, the expression “week” means seven consecutive days. Thus, a three-week period is twenty-one consecutive days starting on any day of the calendar week. The day that the first dose is given is considered to be the first day of the week. Any discussion using calendar weeks is intended to be for illustrative purposes only.

The COMPOUND I may be combined with one or more pharmaceutically acceptable carriers and, optionally, one or more other conventional pharmaceutical adjuvants and administered enterally, e.g. orally, in the form of tablets, capsules, caplets, etc. or parenterally, e.g., intraperitoneally or intravenously, in the form of sterile injectable solutions or suspensions. The enteral and parenteral compositions may be prepared by conventional means.

The infusion solutions according to the present invention are preferably sterile. This may be readily accomplished, e.g. by filtration through sterile filtration membranes. Aseptic formation of any composition in liquid form, the aseptic filling of vials and/or combining a pharmaceutical composition of the present invention with a suitable diluent under aseptic conditions are well known to the skilled addressee.

The COMPOUND I can be used alone or combined with at least one other pharmaceutically active compound for use in these pathologies. These active compounds can be combined in the same pharmaceutical preparation or in the form of combined preparations “kit of parts” in the sense that the combination partners can be dosed independently or by use of different fixed combinations with distinguished amounts of the combination partners, i.e., simultaneously or at different time points. The parts of the kit of parts can then, e.g., be administered simultaneously or chronologically staggered, that is at different time points and with equal or different time intervals for any part of the kit of parts.

Two non-limitative examples of useful compositions are described hereafter.

Composition A:

10.000 100 mg tablets, each comprising 0.2 mg of active ingredient, are prepared:

| | | |
|---|---|---|
| bis(cyanophenyl)methyl-triazole of the formula (I) | 2.00 | g |
| silica, colloidal | 2.00 | g |
| cellulose, microcrystalline | 100.00 | g |
| lactose, spray-dried | 836.00 | g |
| magnesium stearate | 10.00 | g |
| sodium carboxymethylcellulose | 50.00 | g |
| | 1000.00 | g |

All the constituents of the tablet core are mixed together. As soon as a homogeneous mixture is obtained, it is compressed to form tablet cores.

Composition B:

10 000 100 mg tablets, each comprising 0.15 mg of active ingredient, are prepared:

| | | |
|---|---|---|
| bis(cyanophenyl)methyl-triazole of the formula (I) | 1.5 | g |
| lactose, crystalline | 741.50 | g |
| cellulose, microcrystalline | 237.00 | g |
| silica, colloidal | 10.00 | g |
| magnesium stearate | 10.00 | g |
| | 1000 | g |

All the constituents of the tablet core are mixed together. As soon as a homogeneous mixture is obtained, it is compressed to form tablet cores.

Composition C:

10 000, 100 mg tablets, each comprising 0.015 mg of active ingredient, are prepared:

| | | |
|---|---|---|
| bis(cyanophenyl)methyl-triazole of the formula (I) | 0.15 | g |
| lactose, crystalline | 742.85 | g |
| cellulose, microcrystalline | 237.00 | g |
| silica, colloidal | 10.00 | g |
| magnesium stearate | 10.00 | g |
| | 1000 | g |

All the constituents of the tablet core are mixed together. As soon as a homogeneous mixture is obtained, it is compressed to form tablet cores.

Preferred lower dosage tablets may for instance contain 0.05 mg, 0.02 mg, 0.015 mg, 0.01 mg, 0.009 mg, 0.008 mg, 0.007 mg, 0.006 mg, 0.005 mg, 0.004 mg, 0.003 mg, 0.002 mg or 0.001 mg of active ingredient.

However, it should be clearly understood that it is for purposes of illustration only.

The present invention can also be carried out with lower dosage of other aromatase inhibitors such as, anastrazole, megestrol acetate, vorozole, fadrozole, exemestane, aminoglutethimide. The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

The efficacy of COMPOUND I for the prevention of conditions responsive to aromatase inhibition and to inhibition of estrogen biosynthesis, has been demonstrated by the herein-mentioned pharmacological tests. This unexpected pharmacological activity at a lower dosage can also be demonstrated in a clinical study such as the protocol described by Harper-Wynne C and col. (Cancer Epidemiol Biomarkers Prev 2002 Jul; 11(7):614-21). The corresponding content thereof is hereby incorporated by reference.

The pharmacological tests have been carried out by the following pharmacological methods. These tests illustrate the invention without in any way limiting its scope.

1. Aromatase Overexpressing Transgenic Mice and Animal Treatments.

The female aromatase transgenic mice were maintained in a standard colony. Mice were housed in a centralized animal facility accredited by the AAALAC and USDA and maintained according to the recommendations established in the NIH Guide for the Care and Use of Laboratory Animals. Generation of transgenic mice that overexpress aromatase (previously referred to as int-5/aromatase transgenic mice) in mammary glands was previously described (R. R. Tekmal et al., Overexpression of int-5/aromatase in mammary glands of transgenic mice results in the induction of hyperplasia and nuclear abnormalities, Cancer Res. 56 (1996) 3180-3185). All animals were genotyped using Southern blot analysis and age-matched nontransgenic littermates were used as controls in all experiments.

2. Morphological and Histological Assessment of Mammary Glands and other Tissues.

The skin containing the mammary fat pads was fixed in 10% neutral buffered formalin for at least 24 h. The mammary glands were then dissected free from skin and processed for histological examinations (D. Medina, Preneoplastic lesions in mouse mammary tumorigenesis, Methods Cancer Res. 7 (1973) 3-53. 514). Routine section of mammary tissues, uterus and ovary were prepared after fixation by embedding in paraf.n, sectioning at 5 μM, and staining with H&E. To immunolocalize aromatase expression, 5 μM thick sections of aromatase and nontransgenic mammary gland tissue were used. After deparaffination and rehydration in xylene and ethanol, nonspecific sites were blocked by incubating with 1% normal goat serum in 0.05M Tris-HCl buffer. After decanting the reagents, the sections were covered with rabbit polyclonal antibody to rat aromatase synthetic peptide in 1:100 dilution (aromatase antibody was obtained from Dr. Evan Simpson, University of Texas Southwestern Medical Center, Dallas, Tex.; currently at Prince Henry's Institute, Clayton, Vic., Australia). The samples were incubated overnight at 4° C. in a humidied chamber. After three washings with 0.05M Tris-HCl buffer, the slides were incubated with alkaline phosphate conjugated secondary antibody for 30 min at room temperature and washings were repeated and sections were incubated with 1% fast red/naphthol AS phospate solution for 5 min. Sections were washed again and cover slipped and staining pattern was observed using light microscope.

3. Cell Preparation and Characterization

Aromatase transgenic and nontransgenic female littermates from our aromatase colony were the source of mammary tissue. Mammary epithelial and stromal cells were isolated as described before [B. K. Levay-Young, et al. Primary culture systems for mammary biology studies, in: D. Medina (Ed.), Cellular and Molecular Biology of Cancer, Plenum Press, New York, 1987, pp 181-204—F. S. Kittrel, C. J. Obom, D. Medina, Development of mammary paraneoplasia in vivo from mouse mammary epithelial cell lines in vivo, Cancer Res. 52 (1992) 1924-1932.). Mammary glands were dissected from 16 weeks old virgin females, washed in sterile phosphate buffered saline with antibiotics, finely minced with razor blade, and dissociated in gyratory water bath in the presence of 0.15% collagenase (Type A; Boehimger-Mannhem, Indianapolis, Id.) for 4 h at 37° C.

The cell suspension was differentially centrifuged as described before to obtain both epithelial and stromal cells. To establish the purity of epithelial and stromal cells, cell types were identified immunohistochemically using the epithelial and stromal cell specific antibodies. Epithelial and stromal cells were plated into chamber slides and were fixed with Bouin's solution. Cytokeratin and vimentin immunostaining was performed. The stromal cells were immunostained by antibody to vimentin (Zymed) but not by the antibody to cytokeratin confirming their stromal phenotype. Epithelial cells were immunostained by the antibody to cytokeratin (Dako) confirming their epithelial origin.

4. Treatment with Aromatase Inhibitor

Aged (about 16-20 weeks old) virgin aromatase transgenic females were used to investigate the effect of the aromatase inhibitor, COMPOUND I, as a chemopreventive agent against the preneoplastic and neoplastic changes induced in mammary glands of these mice. In this study, age-matched transgenic females were divided into four groups, one group (n=10) served as control group, the other groups received daily s.c. injection of COMPOUND I and three different doses; 0.25, 05, 1.0 μg COMPOUND I per animal in 100 μl of 0.3% hydroxy propyl cellulose (in phospate buffered saline). Control animals were given s.c. injection of vehicle. COMPOUND I was a gift from Drs. Ajay Bhatnagar and Dean Evans of Novartis Pharma (Basel, Switzerland). At the end of the 6-week treatment, mammary glands were removed and one gland each was used for histological analysis. All other glands were pooled then used for biochemical analysis as described below. Along with the mammary glands, uterus and ovaries was collected for biochemical and histological analyses.

5. RNA Analysis

Total RNA from mice mammary and uterine tissue was isolated, following homogenization of the tissue, with the Tri Reagent (Sigma, St. Louis, Mo.) according to the manufacturer's protocol. The specific expression of ER, PR, PCNA, cyclin D1 and other genes was then verified by RT-PCR, using the GeneAmp RNA PCR Kit (Perkin-Elmer, Foster City, Calif.). Use of specific primers and conditions of RT-PCR was described before (N. Kirma, K. Gill, U. Mandava, R. R. Tekmal, Overexpression of aromatase leads to hyperplasia and changes in the expression of genes involved in apoptosis, cell cycle, growth and tumor suppressor function in the mammary glands of the transgenic mice, Cancer Res., 2001, in press). Depending on the abundance of the specific mRNA species, 70 ng-1.0 mg of total RNA was used as starting template in a reverse transcription (RT) reaction mix. The RT-PCR products were visualized on a 1% agarose gel with ethidium bromide staining. To demonstrate that equal amounts of total RNA was used from each sample for estimation of the expression of various genes, glyceraldehyde-3phosphate dehydrogenase (GAPDH), a house keeping gene, was used as invariant control. The densitometric data using the Bio-Rad (Hercules, Calif.) GS-700 imaging densitometer from ethidium bromide staining of RT-PCR products on agarose gels were used for calculating the relative differences in the expression of various mRNA levels in different tissue samples.

6. Western Blot Analysis

To isolate proteins from mammary gland and uterine tissue of both control and COMPOUND I-treated aromatase animals, the tissues were homogenized in lysis buffer. Equal amounts (60 ug) of protein from each sample was separated on a denaturing polyacrylamide gel and transferred to a nylon membrane. Non-speci.c binding of antibodies were blocked by incubation for at least 4 h at room temperature with Tris-buffered saline (TBS) containing 0.05% Triton X-100 (TBST) and 5% nonfat dry milk. Membranes were then incubated with respective primary antibodies (actin, ER, PR, lactoferrin, PCNA and cyclin D1) in TBST-milk overnight at 4° C., and specific binding was visualized by using species-specific (secondary antibodies) IgG followed by enhanced chemiluminescent detection (ECL Kit; Amersham Pharmacia Biotech, N.J.) and exposure to ECL X-ray film. Densitometric data from Western blots (X-ray image of chemiluminescent detection antibody-bound protein) were used for calculating the differences in the expression of individual proteins. Expression of actin, a house keeping protein, was used for normalization as an invariant control if needed.

7. Serum Estradiol and FSH Levels

Blood from both control and COMPOUND I-treated groups was collected by cardiac puncture directly before harvesting tissues. Serum concentrations of estradiol were measured by double antibody radioimmunoassay using commercially available reagents (Diagnostic Products Corp., Los Angeles, Calif.). Using an equivalent of 200 ul of serum in duplicate, the assay had a lower sensitivity of 2.5 pg/ml and an upper limit of 500 pg/ml. Assaying increasing volumes of serum from 50 to 200 μl produced a displacement line parallel to the standard curve. Intra-assay and inter assay coefficients of variation averaged <10.0 and 6.7%, respectively. FSH levels were measured using a standard double antibody radioimmunoassay. Purified FSH obtained from NIH hormone resource was used as a standard. Differences in mean estradiol or FSH levels were compared, and significance was determined using paired Student's t-test.

### RESULTS

Effect of aromatase inhibitor (COMPOUND I) on estrogen-induced hyperplasia in mammary glands, ovary and uterus.

To test whether the preneoplastic/neoplastic changes induced by aromatase in transgenic mice can be completely eliminated or reduced by aromatase inhibitors, we have treated aromatase transgenic females with different doses of the aromatase inhibitor, COMPOUND I (0.25, 0.5, 1.0 and 5 g per day per animal) to completely abrogate or reduce aromatase-induced hyperplastic and other changes in breast tissue. Previous studies have shown that the use of 5 g of COMPOUND I per day per animal (based on tumor reduction) can completely abrogate or reduce aromatase-induced hyperplastic and other changes in breast tissue. Mammary glands from control and various concentrations of COMPOUND I-treated aromatase transgenic animals were examined after completion of 6 weeks of COMPOUND I treatment. Our findings indicate that in contrast to untreated aromatase transgenic females, the aromatase females treated with COMPOUND I for 6 weeks show complete reduction/disappearance of hyperplasia and other preneoplastic and neoplastic changes that were induced by the overexpression of aromatase in these animals. These results clearly demonstrate that breast hyperplasia and other preneoplastic/neoplastic changes that were induced by aromatase overexpression can be abrogated by potent aromatase inhibitors, like COMPOUND I. Our data shows we could completely abrogate aromatase-induced preneoplastic changes by using a low dose of COMPOUND I (0.25 or 0.5 g per day per animal).

The effect of various concentrations of COMPOUND I on mammary gland, ovary and uterine histomorphological features. Histology of mammary gland from control and various concentrations of COMPOUND I-treated aromatase transgenic mice have been carried on. The resulting data shows we could completely abrogate aromatase-induced preneoplastic changes by using a low dose of COMPOUND I (0.25 or 0.5 μg per day per animal). These doses had no effect on uterus and ovary. These low doses of COMPOUND I (0.25 or 0.5 μg per day per animal) had no effect on histo-morphological features of the uterus and ovary.

Effect of COMPOUND I on circulating levels of estradiol in aromatase transgenic mice. No significance difference between control (12.5 pg/ml) and the individual groups treated with low doses of COMPOUND I (0.25 or 0.5 μg per day per animal) in circulating estrogen levels (respectively 12.1 and 12.5 pg/ml). A COMPOUND I dose of 0.5 μg per day per animal leads to a circulating levels of estradiol of 2 pg/ml.

Effect of COMPOUND I on circulating levels of follicle stimulating hormone in aromatase transgenic mice. No significance difference between control and the animals treated with 0.5 μg of COMPOUND I in circulating FSH levels (200 ng/ml). Difference between control and 5.0 μg COMPOUND I-treated groups in circulating FSH levels (480 ng/ml) is very significant (paired Student's t-test).

Effect of COMPOUND I on the protein levels of estrogen receptor (ER), progesterone receptor (PR), cyclin D1 and proliferating cell nuclear antigen (PCNA) in the mammary tissues of aromatase transgenic mice. Densitometric data from Western blot analysis is used for graphical representation after correcting for any difference based on the actin levels. Consistent with decreased mammary proliferation, we have seen decreases in the protein levels of ER, PR, in mammary tissues of COMPOUND I treated animals. We have also noticed no significant change in the levels of PCNA and cyclin D1, also good markers of estrogen-mediated proliferation with low doses of COMPOUND I (0.25 or 0.5 μg per day per animal). With a dose of 5 μg of COMPOUND I the level of PCNA is reduced by 50%. Which is consistent with the observation that low doses of COMPOUND I (0.25 or 0.5 μg per day per animal) had no effect on histo-morphological features of the uterus and ovary.

Effect of COMPOUND I on the protein levels of lactoferrin, ER, and PR in the uterus tissue of aromatase transgenic mice. Densitometric data from Western blot analysis is used for graphical representation after correcting for any difference based on the actin levels. To examine whether the COMPOUND I treatment affected the endocrine functions of these animals, we tested the expression of genes that are under estrogenic regulation In the uterus by Western bolt analysis. The expression of the uterine marker lactoferrin showed no difference between the control and the 0.25- and 0.5 μg-treated animals; however, a 1.8-fold decrease was observed in the 1.0 μg per day per animal-treated animal as compared to the control. No difference was found in the expression of ER and PR in the uterus of the treated animals.

1. Use of bis(cyanophenyl)methyl-triazole or pharmaceutically acceptable salts thereof, for the preparation of a pharmaceutical composition for the prevention of conditions responsive to aromatase inhibition and to inhibition of estrogen biosynthesis in mammals.

2. Use according to claim 1 for inhibiting the development of breast cancer in woman, especially in a post-menopausal woman or a woman predisposed to breast cancer.

3. Use according to claim 1 characterized in that the dosage of bis(cyanophenyl)methyl-triazole or pharmaceutically acceptable salts thereof has no significant effect on the circulating level of estradiol and or circulating level of follicle stimulating hormone in the mammal.

4. A method for preventing conditions responsive to aromatase inhibition and to inhibition of estrogen biosynthesis in mammals comprising administering to a mammal in need of such treatment a therapeutically effective amount of bis(cyanophenyl)methyl-triazole, or pharmaceutically acceptable salts thereof.

5. A method according to claim 4, wherein the mammal is a human, especially a post-menopausal woman or a woman predisposed to breast cancer.

6. A method according to claim 4, for inhibiting the development of breast cancer.

7. A method according to claim 4, wherein the daily dosage of bis(cyanophenyl)methyl-triazole, is 8.3 to 25 fold lower than the daily dosage necessary for the preventive treatment of conditions responsive to aromatase inhibition and to inhibition of estrogen biosynthesis in mammals.

8. A method according to claim 5 wherein the daily dosage of bis(cyanophenyl)methyl-triazole, is 10 to 20 fold lower and preferably with 10 to 16.7 fold lower than the daily dosage necessary for the preventive treatment of such conditions.

9. A method according to claim 4, wherein the daily effective amount of bis(cyanophenyl)methyl-triazole for inhibiting the development of breast cancer in a woman, is 0.1 to 0.3 mg, preferably 0.125 mg to 0.25 mg most preferably 0.15 mg to 0.25 mg of bis(cyanophenyl)methyl-triazole.

10. A method according to claim 4, wherein the daily effective amount of bis(cyanophenyl)methyl-triazole for inhibiting the development of breast cancer in a woman, is 0.001 to 0.099 mg, preferably 0.002 to 0.02 mg of bis(cyanophenyl)methyl-triazole.

11. A method according to claim 4, wherein the daily dosage of bis(cyanophenyl)methyl-triazole has no significant effect on the circulating level of estradiol and or circulating level of follicle stimulating hormone in the mammal.

12. A method according to claim 4, wherein the therapeutically effective amount of bis(cyanophenyl)methyl-triazole is administered to the human subject over at least a three week time period on only about 14.2% to about 42.8% of the days in the time period.

13. A method according to claim 4, wherein the therapeutically effective amount of bis(cyanophenyl)methyl-triazole is administered to the human subject 1 to 3 times a week on alternate days for a period of three weeks or longer.

14. A method according to claim 13, wherein the weekly dosage regimen is carried out for a period of 3, 4, 5, 6, 7, or 8 weeks.

15. A method according to claim 12, wherein the treatment period is followed by a period of one to three weeks, wherein the agent is not administered and this cycle being repeated for from 1 to several cycles.

16. A method according to claim 4, wherein the therapeutically effective amount of bis(cyanophenyl)methyl-triazole is administered to the human subject 7 to 4 times a week or about 100% to about 50% of the days in the time period, for a period of from one to three weeks, followed by a period of one to three weeks, wherein the agent is not administered and this cycle being repeated for from 1 to several cycles.

17. A method according to claim 16, wherein the therapeutically effective amount of bis(cyanophenyl)methyl-triazole is administered to the human subject daily or once each two days.

18. A method according to claim 12, wherein the therapeutically effective amount of bis(cyanophenyl)methyl-triazole has no significant effect on the circulating level of estradiol and/or circulating level of follicle stimulating hormone in the mammal.

19. A method according to claim 12, wherein the effective amount of bis(cyanophenyl)methyl-triazole for inhibiting the development of breast cancer in a woman, is 0.1 to 0.3 mg, preferably 0.125 mg to 0.25 mg most preferably 0.15 mg to 0.25 mg of bis(cyanophenyl)methyl-triazole.

20. A pharmaceutical composition in a unit dosage form containing an amount of bis(cyanophenyl)methyl-triazole or pharmaceutically acceptable salts thereof, 5 to 20 fold lower than the daily dosage necessary for the preventive treatment of conditions responsive to aromatase inhibition and to inhibition of estrogen biosynthesis in mammals.

21. A pharmaceutical composition according to claim 20 wherein the amount of bis(cyanophenyl)methyl-triazole is 8.3 to 25 fold preferably 10 to 20, most preferably 10 to 16.7 fold lower than the daily dosage necessary for the preventive treatment of conditions responsive to aromatase inhibition and to inhibition of estrogen biosynthesis in mammals.

22. A pharmaceutical composition in a unit dosage form according to claim 20 containing an amount of 0.1 to 0.3 mg, preferably 0.125 mg to 0.25 mg most preferably 0.15 mg to 0.25 mg of bis(cyanophenyl)methyl-triazole.

23. A pharmaceutical composition in a unit dosage form containing an amount of bis(cyanophenyl)methyl-triazole or pharmaceutically acceptable salts thereof, characterized in that the amount of bis(cyanophenyl)methyl-triazole has no significant effect on the circulating level of estradiol and or circulating level of follicle stimulating hormone in the mammal.

24. A pharmaceutical composition in a unit dosage form containing an amount of bis(cyanophenyl)methyl-triazole or pharmaceutically acceptable salts thereof, characterized in that the amount of active ingredient is 0.001 to 0.099 mg, preferably 0.002 to 0.02 mg of bis(cyanophenyl)methyl-triazole, for the preventive treatment of conditions responsive to aromatase inhibition and to inhibition of estrogen biosynthesis in mammals.

25. A pharmaceutical composition according to claim 20, for the preventive treatment of breast cancer in woman.

26. An article of manufacture comprising packaging material, and bis(cyanophenyl)methyl-triazole or pharmaceutically acceptable salts thereof, contained within said packaging material, wherein said packaging material comprises label directions which indicate that said bis(cyanophenyl)methyl-triazole, or said pharmaceutically-acceptable salt of bis(cyanophenyl)methyl-triazole, is to be administered to a woman preferably a post-menopausal woman or a woman predisposed to breast cancer, in an amount from about 0.1 mg to about 0.3 mg preferably from about 0.125 mg to about 0.25 mg following a specific dosage regimen to inhibit the development of breast cancer.

27. An article of manufacture comprising packaging material, and bis(cyanophenyl)methyl-triazole or pharmaceutically acceptable salts thereof, contained within said packaging material, wherein said packaging material comprises label directions which indicate that said bis(cyanophenyl)methyl-triazole, or said pharmaceutically-acceptable salt of bis(cyanophenyl)methyl-triazole, is to be administered to a woman preferably a post-menopausal woman or a woman predisposed to breast cancer, in an amount that has no significant effect on the circulating level of estradiol and or circulating level of follicle stimulating hormone in the mammal, following a specific dosage regimen to inhibit the development of breast cancer.

28. An article of manufacture according to claim 27 wherein the effective amount of bis(cyanophenyl)methyl-triazole is in the form of a pharmaceutical composition as described in claim 20.
